(19) United States
(12) Patent Application Publication
(10) Pub. No.: US 2008/0193403 A1
(43) Pub. Date: Aug. 14, 2008

(54) Inducing And Maintaining Hair Color

(75) Inventor: Mark L. Witten, Tucson, AZ (US)

Correspondence Address: JONES DAY, 222 EAST 41ST ST, NEW YORK, NY, 10017, US

(73) Assignee: IMMUNEREGEN BIOSCIENCES, INC, SCOTTSDALE, AZ (US)

(21) Appl. No.: 11/587,594

(22) PCT Filed: Apr. 18, 2005
(86) PCT No.: PCT/US2005/013112

§ 371 Date: Mar. 14, 2008

Related U.S. Application Data
(60) Provisional application No. 60/565,021, filed on Apr. 26, 2004.

(51) Int. Cl. A61K 8/64; A61K 38/08; A61Q 5/06

(52) U.S. Cl. 424/70.6; 514/15

(57) ABSTRACT

Hair pigment loss is treated by administration of Substance P or a bioactive analog thereof. The subject may have pigment loss in hair due to any of a variety of reasons, including drug side effects, genetic composition, aging, or nutritional deficit. The treatment can be conveniently administered topically. Maintenance or increasing the amount of hair color results from the treatments.

This application claims the benefit of provisional application Ser. No. 60/565,021 filed Apr. 26, 2004, the disclosure of which is expressly incorporated herein.

### TECHNICAL FIELD OF THE INVENTION

This invention is related to the area of hair coloration. In particular, it relates to preventing and/or reversing hair pigment loss.

### BACKGROUND OF THE INVENTION

Hair or pili are fine threadlike appendages of the skin which normally cover the entire body (with the exception of the palms of the hands and soles of the feet, and the flexor surfaces of joints). A hair comprises a root embedded in a hair follicle and a free portion (the stem or shaft). The term hair refers to both mature hair as well as the soft, downy hair known as vellus hair.

The hair bulb or follicle is a compact structure located in the dermis layer of the skin and is composed of three main cellular groups. The first comprises a compact group of fibroblasts known as the dermal papilla which includes a capillary system. The second group comprises germinative epithelial cells of the hair bulb which proliferate and differentiate to give rise to the mature hair shaft. The third group of cells are fibroblasts which exist around the outside of the bulb in the connective tissue sheath. Recently, unpigmented melanocyte stem cells were discovered within the hair follicle, in particular within the lower permanent portion of the follicle. In contrast, differentiated melanocytes reside in the hair bulb at the base of the transient portion of the hair follicle.

During the pilar cycle, the hair forms, grows, and falls out before being replaced by a new hair which appears in the same follicle. During a pilar cycle, three phases are successively observed, namely, the anagen phase, the catagen phase and the telogen phase. During the anagen phase, the hair undergoes a period of active growth associated with intensive metabolic activity in the bulb. The anagen phase lasts for about two to five years. The catagen phase is transitory (from days to weeks) and is marked by a slowing-down of mitotic activity. During this phase, the hair undergoes involution and the follicle atrophies. The telogen phase corresponds to a period of rest of the follicle and shedding of the hair. It lasts for a few months. The old hair is pushed by an incipient anagen hair. A head of hair is thus constantly renewed and, of the approximately 150,000 hairs which a head of hair contains, at any time approximately 10% of them are at rest and will therefore be replaced in a few months.

Hair is colored by a pigment system that involves melanocytes. Melanin is produced by the melanocytes in the matrix area of the follicle. The melanin is incorporated into differentiating matrix cells by phagocytosis and becomes part of the hair shaft. Hair pigmentation may change due to age, disease, injury, or side effects of drugs. When pigmentation changes prematurely, it can be due, for example, to genetic factors. Vitamin B deficiency, and side effects of drugs for treatment of arthritis.

There is a continuing need in the art for substances which suppress, reduce, prevent, or reverse the graying process of hair.

### SUMMARY OF THE INVENTION

According to the invention a method is provided for increasing the ratio of pigmented to non-pigmented hairs on a non-bald person in need thereof. An agent is applied to the scalp of the person. The scalp comprises one or more non-pigmented hairs. The agent is selected from the group consisting of substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met (0₂)¹¹]-substance P, and analogs having the amino acid backbone RPKPQQFFGLM-NH₂ (SEQ ID NO: 1). The ratio of pigmented to non-pigmented hairs on the scalp is thereby increased.

According to the invention a method is provided for increasing or maintaining the number of melanocyte stem cells in the scalp of a non-bald person. An agent is applied to the scalp of the person. The agent is selected from the group consisting of substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met (0₂)¹¹]-substance P, and analogs having the amino acid backbone RPKPQQFFGLM-NH₂ (SEQ ID NO: 1). The number of melanocyte stem cells on the scalp is thereby increased or maintained.

According to the invention a method is provided for maintaining or inducing hair color in a non-bald person. An agent is applied to the scalp of the person. The agent is selected from the group consisting of substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met (0₂)¹¹]-substance P, and analogs having the amino acid backbone RPKPQQFFGLM-NH₂ (SEQ ID NO: 1). The amount of hair color on the scalp of the non-bald person is thereby maintained or increased.

These and other embodiments which will be apparent to those of skill in the art upon reading the specification provide the art with reagents and methods for treating hair pigmentation loss.

### DETAILED DESCRIPTION OF THE INVENTION

The inventors have discovered that Substance P (SP) and bioactive analogs thereof can induce or maintain hair pigmentation. This discovery can be applied to any of the many diverse causes of hair color loss, including but not limited to age, disease, injury, traumatic shock, genetic factors, vitamin B deficiency, or side effects of drugs, such as cyclophosphamide.

Preferably the effective agent of the present invention is applied topically. This can be accomplished in a liquid, gel, lotion, ointment, aerosol or other convenient medium. However, other means, as are known in the art, such as intravenous, subcutaneous, intramuscular, intraperitoneal, transdermal, and intra-arterial administration can be used as alternatives. Any such means as is known in the art can be used.

The person being treated can be one who has lost pigmentation or who is expected to lose pigmentation in his or her hair. Typically the person does not also have bald areas on the scalp, has not been treated with irradiation, does not have alopecia, and is not undergoing chemotherapy. Persons who do not have bald areas on their scalps or are not experiencing a treatment or a disease which causes hair loss are collectively referred to herein as “non-bald persons.” While the applicant does not intend to be bound by any proposed mechanism of action of the present invention, it is believed that the effective agent inhibits PARP (poly-ADP-ribose polymerase) expression which inhibits apoptosis of melanocyte stem cells. Inhibition of apoptosis causes the melanocyte stem cells to be maintained, i.e., renewed.

Substance P (RPKPQQFFGLM-NH₂; SEQ ID NO: 1) or any of its bioactive analogues can be used in the methods of the present invention. These include, but are not limited to: [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar, Met(0₂)¹¹]-substance P, and other analogs which have the amino acid backbone RPKPQQFFGLM-NH₂ (SEQ ID NO: 1). Bioactive analogs according to the invention are those which act as competitive inhibitors of SP by binding to the SP receptors (NK-1, NK-2, or NK-3 receptors). Other derivatives as are known in the art and commercially available (e.g., from Sigma, St. Louis, Mo. or from Polypeptide Laboratories A/S, Hillerod, Denmark) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on SP while retaining the peptide backbone. Again, routine testing will determine which of such modifications do not adversely affect biological activity.

Typical concentration ranges of substance P or its bioactive analogue in the aerosol administered is between 0.001 and 50 μM. Concentrations in the range of between 0.05 and 5 μM are particularly useful. It can be advantageously administered as a liquid at a concentration between about 0.1 and 10 μM. Total doses of 0.5 μg to 100 μg can be administered daily. Other dosing regimens can be readily determined and used.

The method of the present invention is useful in the treatment of canities in mammals, and as such may be used to promote, increase, or assist in the maintenance or reacquisition of hair pigment. Subjects may be male or female. Successful treatment results in an increased ratio of pigmented to non-pigmented hairs, an increased number of melanocyte stem cells, or an increased amount of hair color pigment on the scalp overall. The pigment per hair may be increased, or the number of pigmented hairs may be increased, or both parameters may be increased. Successful treatment also includes maintenance of the ratio of pigmented to non-pigmented hairs, maintenance of the number of melanocyte stem cells, and maintenance of the amount of hair color per hair or per head of hair.

Subjects to be treated according to the invention include human subjects as well as other mammalian subjects, such as dogs, cats, mice, rats, goats, llamas, minks, seals, beavers, ermines, and sheep. These can be treated for loss or diminution of hair pigmentation in order to enhance wool or pelt color.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE

[Sar⁹, Met(0₂)¹¹]-substance P was formulated in a hair styling gel at a concentration of 0.001 mg/ml, and approximately 2 mls were applied daily to my scalp. After a few months, I perceived that the overall color of my hair was becoming more like my original brown color, i.e., a higher percentage of the hairs are brown than before I started applying the substance P analog. I estimate that my hair is 40% darker than previously. This observation is consistent with results observed on radiation-exposed mice that had been given the substance P analog. The mice regrew hair that was lost upon radiation exposure and the new growth was darker than in the non-exposed area.

1. A method of increasing the ratio of pigmented to non-pigmented hairs on a non-bald person in need thereof, comprising:
applying an agent to the scalp of the person, wherein the scalp comprises one or more non-pigmented hairs, whereby the ratio of pigmented to non-pigmented hairs on the scalp increases, wherein the agent is selected from the group consisting of substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met (0₂)¹¹]-substance P, and analogs of substance P having the amino acid backbone RPKPQQFFGLM-NH₂ (SEQ ID NO: 1).

2. A method of increasing or maintaining the number of melanocyte stem cells in the scalp of a non-bald person, comprising:
applying an agent to the scalp of the person, whereby the number of melanocyte stem cells in the scalp is maintained or increased, wherein the agent is selected from the group consisting of substance P, [Met-OH¹¹]-substance P, [Met-OMe¹¹]-substance P, [Nle¹¹]-substance P, [Pro⁹]-substance P, [Sar⁹]-substance P. [Tyr⁸]-substance P, [p-Cl-Phe^{7,8}]-substance P, [Sar⁹,Met (0₂)¹¹]-substance P, and analogs of substance P having the amino acid backbone RPKPQQFFGLM-NH₂ (SEQ ID NO: 1).

3. A method of maintaining or inducing hair color in a non-bald person, comprising:
applying an agent to the scalp of the person, whereby scalp hair color is maintained or increased, wherein the agent is selected from the group consisting of substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met (0₂)¹¹]-substance P, and analogs of substance P having the amino acid backbone RPKPQQFFGLM-NH₂ (SEQ ID NO: 1).

4. The method of claim 1, 2, or 3 wherein the agent is applied topically.

5. The method of claim 1, 2, or 3 wherein the agent is applied daily.

6. The method of claim 1, 2, or 3 wherein the agent is applied in a liquid.

7. The method of claim 1, 2, or 3 wherein the agent is administered in a gel.

8. The method of claim 1, 2, or 3 wherein the agent is applied in an aerosol.

9. The method of claim 1, 2, or 3 wherein the agent is administered in a lotion or ointment.

10. The method of claim 1, 2, or 3 wherein the agent is [Sar⁹, Met (0₂)¹¹]-substance P.

11. The method of claim 1, 2, or 3 wherein the agent is Substance P.

12. The method of claim 1, 2, or 3 wherein the agent is a Substance P analog which has the amino acid backbone RPKPQQFFGLM-NH₂ (SEQ ID NO: 1).

13. The method of claim 1, 2, or 3 wherein the amount administered per dose is between 0.5 μg and 100 μg.


SEQUENCE LISTING (1 sequence)

SEQ ID NO: 1 - protein, 11 aa, Homo sapiens
RPKPQQFFGLM